(19) United States
(12) Patent Application Publication
(10) Pub. No.: US 2025/0161297 A1
(43) Pub. Date: May 22, 2025

(54) COMPOSITIONS AND METHODS FOR INHIBITING FSP1

(71) Applicants: THE REGENTS OF THE UNIVERSITY OF CALIFORNIA, Oakland, CA (US); CZ BIOHUB SF, LLC, San Francisco, CA (US)

(72) Inventors: James Arthur Olzmann, Oakland, CA (US); Kirill G. Bersuker, Hayward, CA (US); Julia Sabine Schaletzky, El Cerrito, CA (US); Edward Eric Wehri, Oakland, CA (US); Joseph Michael Hendricks, Berkeley, CA (US); Cody Edward Doubravsky, Oakland, CA (US)

(21) Appl. No.: 18/838,647

(22) PCT Filed: Feb. 28, 2023
(86) PCT No.: PCT/US23/14034

§ 371 Date: Aug. 15, 2024

Related U.S. Application Data
(60) Provisional application No. 63/430,381, filed on Dec. 6, 2022.
(60) Provisional application No. 63/314,552, filed on Feb. 28, 2022.

(51) Int. Cl. A61K 31/496; A61K 31/429; A61K 31/433; A61K 31/4375; A61K 31/454; A61K 31/4725; A61K 31/55; A61P 35/00

(52) U.S. Cl. CPC A61K 31/496; A61K 31/429; A61K 31/433; A61K 31/4375; A61K 31/454; A61K 31/4725; A61K 31/55; A61P 35/00

(57) ABSTRACT

Provided herein are methods of inhibiting FSP1 and using FSP1 inhibitors as therapeutics for cancer.

### STATEMENT OF GOVERNMENT SUPPORT

This invention was made with government support under GM112948 awarded by the National Institutes of Health. The government has certain rights in the invention.

### FIELD

The present disclosure relates generally to methods for treating cancers by regulating the AIFM2 gene and inhibiting FSP1.

### BACKGROUND

Ferroptosis is an iron-dependent, non-apoptotic form of regulated cell death that is characterized by the accumulation of oxidatively damaged phospholipids (i.e. lipid peroxides) (see, e.g., Dixon, S. J. et al. Cell 149, 1060-1072 (2012); Bersuker, K. et al. Nature 575, 688-692 (2019)). The selenoprotein glutathione (GSH) peroxidase 4 (GPX4) provides the primary cellular mechanism of protection against ferroptosis, catalyzing the detoxification of lipid peroxides by converting them into non-toxic lipid alcohols (see, e.g., Angeli, J. P. F et. al. Trends Pharmacol. Sci. 38, 489-498 (2017); Jiang, X. et. al. Nat. Rev. Mol. Cell Biol. 22, 266-282 (2021)) Small molecule inhibitors targeting the GSH-GPX4 pathway have played pivotal roles in the discovery and characterization of ferroptosis, including system xc-inhibitors (erastin (Dixon et. al. 2012) and imidazole ketone erastin (Zhang, Y. et al. Cell Chem. Biol. 26, 623-633 (2019)) and GPX4 inhibitors (RSL3 (Yang, W. S. et. al. Chem. Biol. 15, 234-245 (2008)), ML162, and ML210). Ferroptosis has been implicated in the cell death and dysfunction in degenerative diseases and triggering ferroptosis by inhibition of the GSH-GPX4 pathway has emerged as a promising strategy to trigger cell death in cancer, showing efficacy in preclinical models of drug-resistant persistent cancer cells that give rise to relapse (Dixon et. al. 2012; Dixon et. al. Annu. Rev. Cancer Biol. 3, 35-54 (2019); Stockwell, B. R. et. al. Cell 171, 273-285 (2017)) as well as a variety of difficult to treat cancers such as pancreatic ductal adenocarcinoma (PDAC) (Badgley, M. A. et al. Science 368, 85-89 (2020)) and MYCN-amplified neuroblastoma (Floros, K. V. et al. Cancer Res. 81, 1896-1908 (2021); Lu, Y. et al. Cell Death Dis. 12, 511 (2021)).

Despite the data supporting the clinical relevance of ferroptosis for cancer treatment, recent findings have uncovered protective mechanisms that promote resistance to ferroptosis inducing agents targeting the GSH-GPX4 pathway. The oxidoreductase FSP1 mediates a GSH-independent ferroptosis suppression pathway. In this pathway, myristoylated FSP1 recruited to the plasma membrane catalyzes the reduction extra-mitochondrial Coenzyme Q10, generating the antioxidant form of Coenzyme Q10 that blocks the propagation of lipid peroxides (Bersuker et. al. 2019; Doll, S. et al. Nature 575, 693-698 (2019)). Moreover, FSP1 gene expression strongly correlates with resistance to GPX4 inhibitors, indicating that FSP1 expression is a biomarker of ferroptosis resistance that predicts the efficacy of GPX4 inhibitors in triggering ferroptosis in cancer, and genetic disruption of FSP1 sensitizes cancer cells to ferroptosis and slows tumor growth when combined with the loss of GPX4. These findings establish FSP1 as a key ferroptosis resistance factor that compensates for the loss of GPX4 (Bersuker et. al. 2019; Doll et. al. 2019) and suggest that inhibitors of FSP1 would be effective therapeutic agents when used in combination with ferroptosis inducers targeting the GSH-GPX4 pathway (Bersuker et. al. 2019; Zhang et. al. 2019; Hangauer, M. J. et. al. Nature 551, 247-250 (2017); Rashidipour, N. et al. Toxicology 152407 (2020); Brea-Calvo, G. Free Radic. Biol. Med. 40, 1293-1302 (2006)). Consistent with this possibility, an FSP1 inhibitor-iFSP1-sensitized multiple cancer cell lines to ferroptosis triggered by GPX4 inhibition. iFSP1 provides important proof of concept, but additional structurally distinct FSP1 inhibitors that are more potent and that are active in vivo are needed to explore the potential of FSP1 as a therapeutic target.

Thus, a need exists for FSP1 inhibitors and their use in cancer therapy.

### SUMMARY

Provided herein are methods of inhibiting ferroptosis suppressor protein 1 (FSP1) comprising contacting FSP1 with an FSP1 inhibitor in an amount effective to inhibit FSP1, wherein the FSP1 inhibitor is a compound as shown in Table A below.

Also provided are methods for treating cancer in a subject suffering therefrom comprising administering to the subject a therapeutically effective amount of an FSP1 inhibitor, wherein the FSP1 inhibitor is a compound as shown in Table A. In some cases, the FSP1 inhibitor is selected from the following compounds:

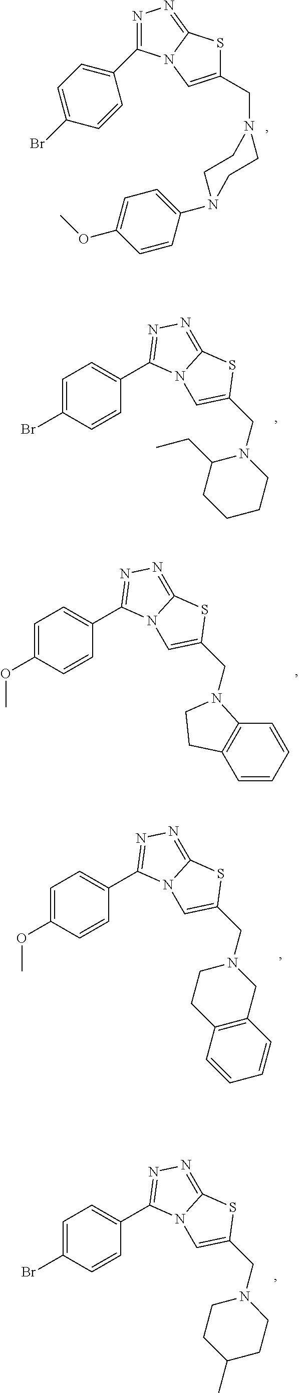 [SMILES: COc1ccc(N2CCN(Cc3cn4c(-c5ccc(Br)cc5)nnc4s3)CC2)cc1 ; CCC1CCCCN1Cc1cn2c(-c3ccc(Br)cc3)nnc2s1 ; COc1ccc(-c2nnc3sc(CN4CCc5ccccc54)cn23)cc1 ; COc1ccc(-c2nnc3sc(CN4CCc5ccccc5C4)cn23)cc1 ; CC1CCN(Cc2cn3c(-c4ccc(Br)cc4)nnc3s2)CC1]

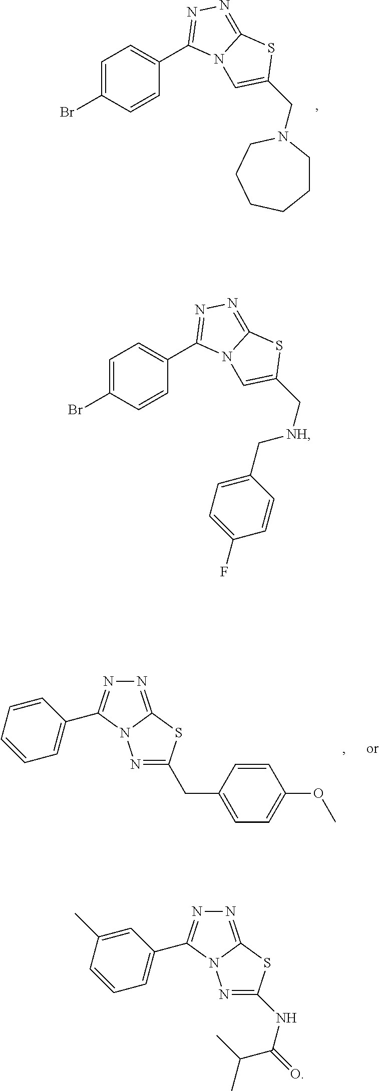

In some cases, the methods also comprise administering to the subject a second therapeutic agent or regimen. In some embodiments, the second therapeutic agent or regimen comprises radiation, photodynamic therapy, a ferroptosis inducer, including a GPX4 inhibitor, a system xc-inhibitor, an inhibitor of glutathione synthesis, or an endoperoxide. In some cases, the GPX4 inhibitor is DPI3, DPI4, DPI6, DPI7, DPI8, DPI9, DPI10, DPI12, DPI13, DPI15, DPI17, DPI18, DPI19, FIN56, JKE-1674, JKE-1716, ML162, ML210, RSL3, FINO2, Altretamine, NSC144988, or Withaferin A. In some cases, the system xc-inhibitor is erastin, erastin2, imidazole ketone erastin, piperazine erastin, DPI2, RSL5, glutamate, sulfasalazine, or sorafenib. In some cases, the inhibitor of glutathione synthesis is buthionine sulfoximine or cyst(e)inase. In some cases, the endoperoxide is artemisinin, dihydroartemisinin (DHA), artemether, arteether, artesunate, artelininic acid, artesunate, artelinate, artemisone, 3-artesanilide, artefenomel, FINO2, or FINO3.

In some embodiments, the cancer is a ferroptosis-resistant cancer or a GPX4-inhibitor-resistant cancer. In some cases, the cancer is one which expresses FSP1. In some cases, the cancer is lung cancer, liver cancer, glial cancer, bone cancer, connective tissue cancer, pancreatic cancer, neuroblastoma, colorectal cancer, astrocytoma, adenocarcinoma, breast cancer, brain cancer, liver cancer, kidney cancer, skin cancer, or intestinal cancer.

In some embodiments, the subject had previously undergone a prior therapy, prior to administration of the FSP1 inhibitor. In some cases, the prior therapy was one that triggers oxidative lipid damage. In some cases, the prior therapy was one or more of photodynamic therapy, radiation, treatment with a GPX4 inhibitor, treatment with an inhibitor of glutathione synthesis, treatment with a system xc-inhibitor, or treatment with an ubiquinone synthesis pathway inhibitor.

### BRIEF DESCRIPTION OF THE DRAWINGS

FIG. 1 shows the structures of several FSP1 inhibitors identified (FSEN1-19) as well as their IC50 for inhibition of FSP1 activity in vitro and their EC50 for triggering ferroptosis in H460^{C} GPX4KO cells.

FIG. 2 shows A) the activity of FSEN1, alone or in combination with the GPX4-inhibitors ML162 or RSL3 against H460^{C} cells; B) the activity of FSEN1 alone or in the presence of various inhibitors of ferroptosis (ferrostatin, DFO, idebenone, tocopherol), apoptosis (Z-VAD), and necroptosis (Nec1s) against H460^{C} cells; C) the activity of FSEN1 alone or in combination with RSL3 in H460° control and FSP1KO cells.

FIG. 3 shows that FSEN1 sensitizes cancer cells from different origins to ferroptosis, A) Dose response of RSL3-induced cell death in the indicated cancer cell lines treated in the presence and absence of 1 μM FSEN1 and 2 μM Fer-1 as indicated. Data are mean±SEM from three biological replicates; and B) Quantification of cell death in melanoma cell lines treated as in (A), calculated as SYTOX green positive object per mm². Data are mean±SEM of two biological replicates.

FIG. 4 shows activity inducers of ferroptosis with FSEN1: A, B) Dose response of DHA and FINO2-induced cell death in H460^{C} Cas9 (A) and FSP1^{KO} (B) cells co-treated with vehicle or 1 μM FSEN1. Data are mean±SEM bars from two biological replicates. C,D) Dose response of DHA-induced cell death in H460^{C} cells co-treated with vehicle (C) or 1 μM FSEN1 (D) together with the indicated inhibitors of ferroptosis (Fer-1 [2 μM], DFO [100 μM], idebenone [10 μM], tococopherol [10 μM]), apoptosis (Z-VAD [10 μM]), and necroptosis (Nec1s [1 μM]). Data are mean±SEM from two biological replicates.

### DETAILED DESCRIPTION

Induction of ferroptosis has emerged as a promising strategy to treat therapy-resistant cancer cells. Despite the discovery of multiple cellular ferroptosis defense systems, current ferroptosis inducers are mostly limited to the GSH-GPX4 pathway, impeding the study and assessment of other ferroptosis regulators as therapeutic targets. Described herein is the discovery and characterization of structurally distinct small molecule FSP1 inhibitors that directly inhibit FSP1 activity and sensitize cancer cells to ferroptosis. The most potent of these new FSP1 inhibitors, FSEN1, is an uncompetitive FSP1 inhibitor that exhibits synthetic lethality with GPX4 inhibitors. FSEN1 is synthetic lethal with endoperoxide-containing ferroptosis inducers, including FINO2 and the FDA approved compound DHA.

Emerging data indicate that several different types of cancer are sensitive to ferroptosis induced by inhibition of the GSH and GPX4 ferroptosis suppression pathway. Given the observation that FSP1 expression correlates with ferroptosis resistance (Bersuker et. al. 2019; Doll et. al. 2019), it is likely that FSEN1 could further sensitize many cancer types to ferroptosis inducers targeting the GSH-GPX4 axis. The difference in the amount of sensitization FSEN1 has on different cancer cell lines indicates that some cancers are more reliant on FSP1 for ferroptosis suppression than others. Strong sensitization was observed in H460 and A549 cancer cells, which are both lung cancer cell lines with KEAP1 mutations. KEAP1 regulates a canonical pathway that mediates the ubiquitin-dependent proteasomal clearance of NRF2, a master transcriptional factor that governs expression of antioxidant factors and is known to confer ferroptosis resistance (Sun, X. et. al. 2016; Shin, D. et. al. Free Radic. Biol. Med. 129, 454-462 (2018); Takahashi, N. et al. Mol. Cell 80, 828-844 (2020)). These cell lines have high FSP1 and low GPX4 protein levels, suggesting a means to stratify cancers that will respond more strongly than others to FSP1 inhibition based upon FSP1 and GPX4 levels. A similar increased effectiveness of DHODH inhibition on ferroptosis induction in cancer cells with low GPX4 was previously observed (Mao, et al. (2021). Nature 593, 586-590).

Disclosed herein are FSP1 inhibitors, having structures as shown in Table A:

TABLE A

| FSEN-No. | Structure |
|---|---|
| FSEN-1 | 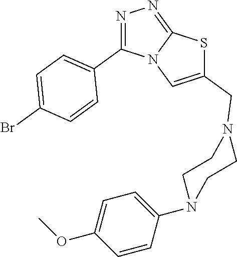 [SMILES: COc1ccc(N2CCN(Cc3cn4c(-c5ccc(Br)cc5)nnc4s3)CC2)cc1] |
| FSEN-2 | 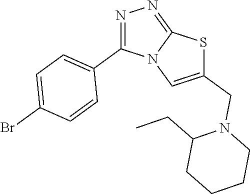 [SMILES: CCC1CCCCN1Cc1cn2c(-c3ccc(Br)cc3)nnc2s1] |
| FSEN-3 | 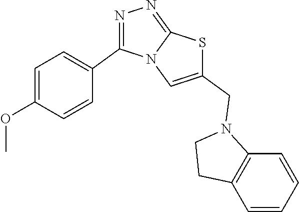 [SMILES: COc1ccc(-c2nnc3sc(CN4CCc5ccccc54)cn23)cc1] |
| FSEN-4 | 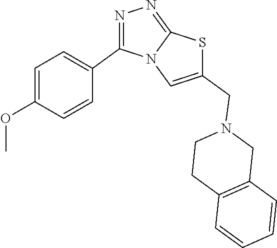 [SMILES: COc1ccc(-c2nnc3sc(CN4CCc5ccccc5C4)cn23)cc1] |
| FSEN-5 | 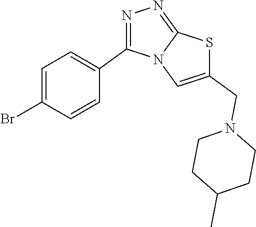 [SMILES: CC1CCN(Cc2cn3c(-c4ccc(Br)cc4)nnc3s2)CC1] |
| FSEN-6 | 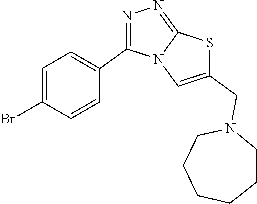 [SMILES: Brc1ccc(-c2nnc3sc(CN4CCCCCC4)cn23)cc1] |
| FSEN-7 | 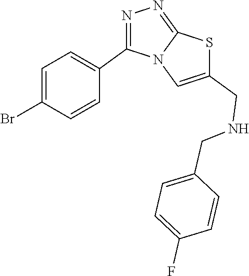 [SMILES: Fc1ccc(CNCc2cn3c(-c4ccc(Br)cc4)nnc3s2)cc1] |
| FSEN-8 | 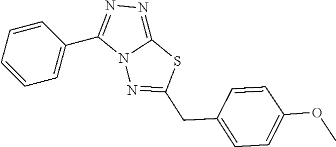 [SMILES: COc1ccc(Cc2nn3c(-c4ccccc4)nnc3s2)cc1] |
| FSEN-9 | 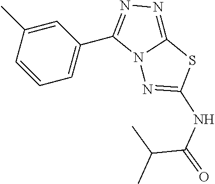 [SMILES: Cc1cccc(-c2nnc3sc(NC(=O)C(C)C)nn23)c1] |
| FSEN-10 | 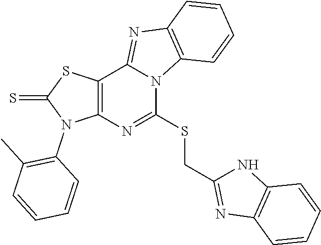 [SMILES: Cc1ccccc1-n1c(=S)sc2c1nc(SCc1nc3ccccc3[nH]1)n1c3ccccc3nc21] |
| FSEN-11 | 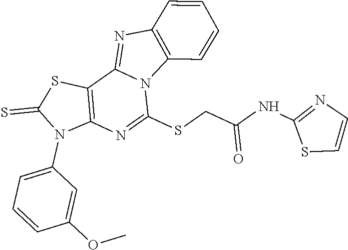 [SMILES: COc1cccc(-n2c(=S)sc3c2nc(SCC(=O)Nc2nccs2)n2c4ccccc4nc32)c1] |
| FSEN-12 | 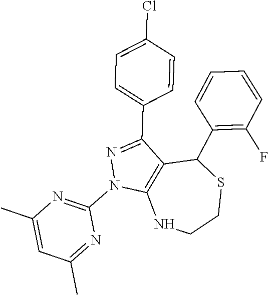 [SMILES: Cc1cc(C)nc(-n2nc(-c3ccc(Cl)cc3)c3c2NCCSC3c2ccccc2F)n1] |
| FSEN-13 | 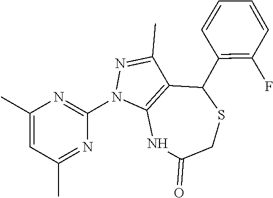 [SMILES: Cc1cc(C)nc(-n2nc(C)c3c2NC(=O)CSC3c2ccccc2F)n1] |
| FSEN-14 | 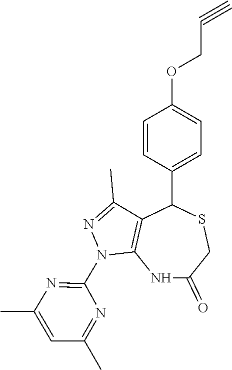 [SMILES: C#CCOc1ccc(C2SCC(=O)Nc3c2c(C)nn3-c2nc(C)cc(C)n2)cc1] |
| FSEN-15 | 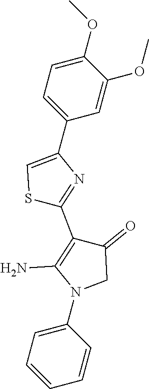 [SMILES: COc1ccc(-c2csc(C3=C(N)N(c4ccccc4)CC3=O)n2)cc1OC] |
| FSEN-16 | 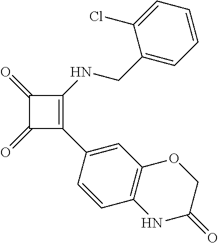 [SMILES: O=C1COc2cc(-c3c(NCc4ccccc4Cl)c(=O)c3=O)ccc2N1] |
| FSEN-17 | 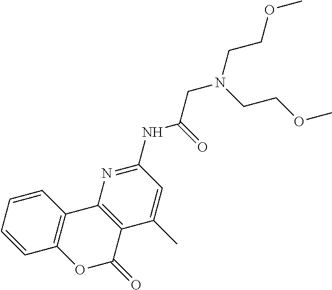 [SMILES: COCCN(CCOC)CC(=O)Nc1cc(C)c2c(=O)oc3ccccc3c2n1] |
| FSEN-18 | 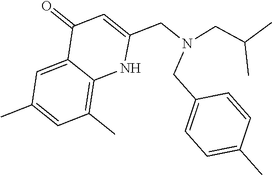 [SMILES: Cc1ccc(CN(Cc2cc(=O)c3cc(C)cc(C)c3[nH]2)CC(C)C)cc1] |
| FSEN-19 | 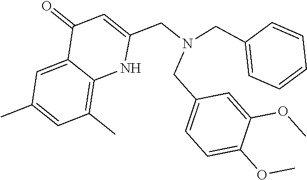 [SMILES: COc1ccc(CN(Cc2ccccc2)Cc2cc(=O)c3cc(C)cc(C)c3[nH]2)cc1OC] |
| FSEN-20 | 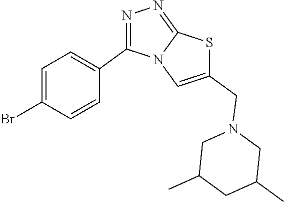 [SMILES: CC1CC(C)CN(Cc2cn3c(-c4ccc(Br)cc4)nnc3s2)C1] |
| FSEN-21 | 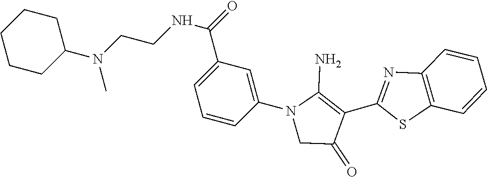 [SMILES: CN(CCNC(=O)c1cccc(N2CC(=O)C(c3nc4ccccc4s3)=C2N)c1)C1CCCCC1] |
| FSEN-22 | 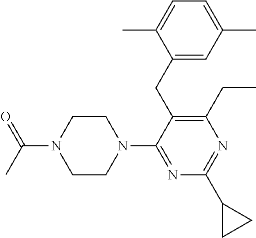 [SMILES: CCc1nc(C2CC2)nc(N2CCN(C(C)=O)CC2)c1Cc1cc(C)ccc1C] |
| FSEN-23 | 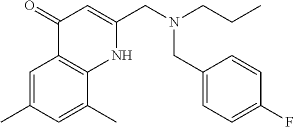 [SMILES: CCCN(Cc1ccc(F)cc1)Cc1cc(=O)c2cc(C)cc(C)c2[nH]1] |
| FSEN-24 | 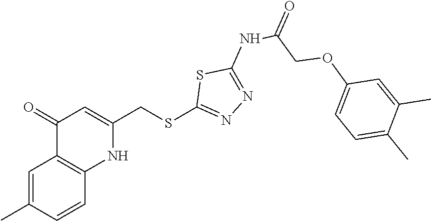 |
| FSEN-25 | 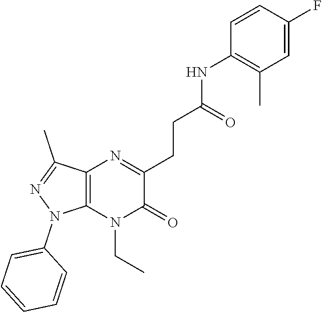 [SMILES: CCn1c(=O)c(CCC(=O)Nc2ccc(F)cc2C)nc2c(C)nn(-c3ccccc3)c21] |
| FSEN-26 | 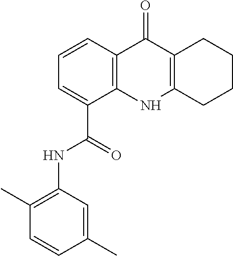 [SMILES: Cc1ccc(C)c(NC(=O)c2cccc3c(=O)c4c([nH]c23)CCCC4)c1] |
| FSEN-27 | 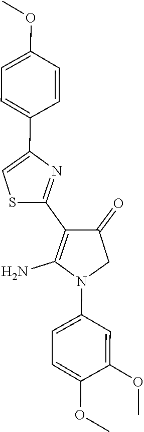 [SMILES: COc1ccc(-c2csc(C3=C(N)N(c4ccc(OC)c(OC)c4)CC3=O)n2)cc1] |
| FSEN-28 | 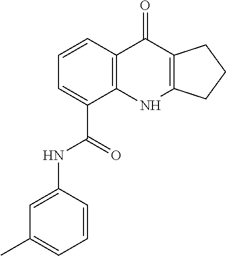 [SMILES: Cc1cccc(NC(=O)c2cccc3c(=O)c4c([nH]c23)CCC4)c1] |
| FSEN-29 | 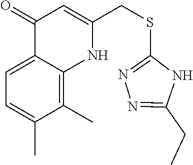 [SMILES: CCc1nnc(SCc2cc(=O)c3ccc(C)c(C)c3[nH]2)[nH]1] |
| FSEN-30 | 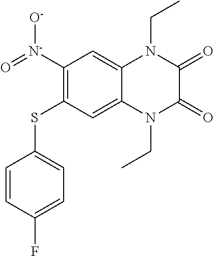 |
| FSEN-31 | 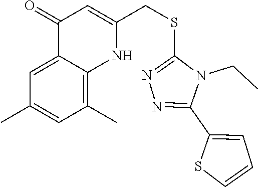 [SMILES: CCn1c(SCc2cc(=O)c3cc(C)cc(C)c3[nH]2)nnc1-c1cccs1] |
| FSEN-32 | 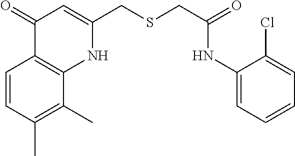 [SMILES: Cc1ccc2c(=O)cc(CSCC(=O)Nc3ccccc3Cl)[nH]c2c1C] |
| FSEN-33 | 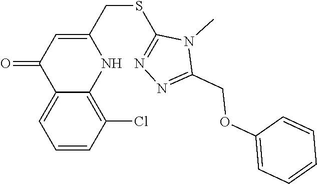 [SMILES: Cn1c(COc2ccccc2)nnc1SCc1cc(=O)c2cccc(Cl)c2[nH]1] |
| FSEN-34 | 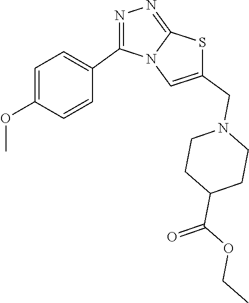 [SMILES: CCOC(=O)C1CCN(Cc2cn3c(-c4ccc(OC)cc4)nnc3s2)CC1] |
| FSEN-35 | 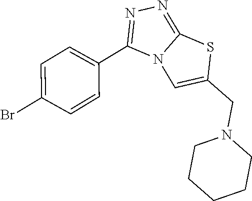 [SMILES: Brc1ccc(-c2nnc3sc(CN4CCCCC4)cn23)cc1] |
| FSEN-36 | 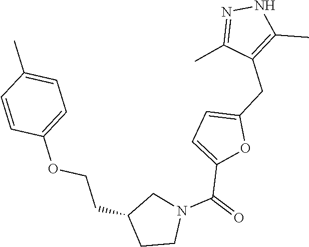 [SMILES: Cc1ccc(OCC[C@H]2CCN(C(=O)c3ccc(Cc4c(C)n[nH]c4C)o3)C2)cc1] |
| FSEN-37 | 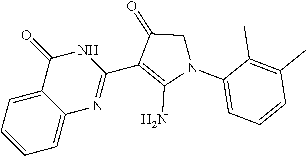 [SMILES: Cc1cccc(N2CC(=O)C(c3nc4ccccc4c(=O)[nH]3)=C2N)c1C] |
| FSEN-38 | 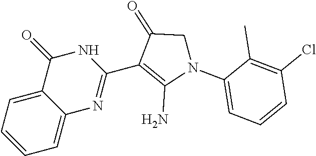 [SMILES: Cc1c(Cl)cccc1N1CC(=O)C(c2nc3ccccc3c(=O)[nH]2)=C1N] |
| FSEN-39 | 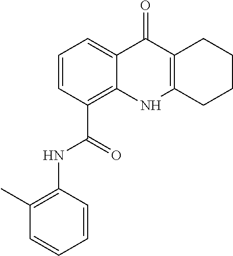 [SMILES: Cc1ccccc1NC(=O)c1cccc2c(=O)c3c([nH]c12)CCCC3] |
| FSEN-40 | 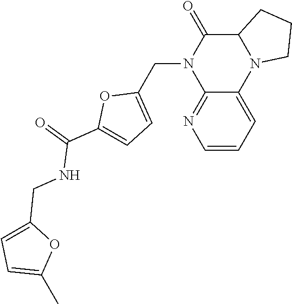 [SMILES: Cc1ccc(CNC(=O)c2ccc(CN3C(=O)C4CCCN4c4cccnc43)o2)o1] |
| FSEN-41 | 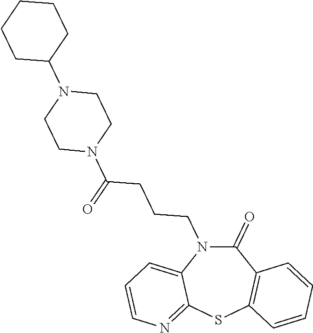 [SMILES: O=C(CCCN1C(=O)c2ccccc2Sc2ncccc21)N1CCN(C2CCCCC2)CC1] |
| FSEN-42 | 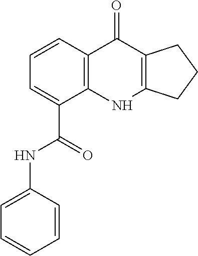 [SMILES: O=C(Nc1ccccc1)c1cccc2c(=O)c3c([nH]c12)CCC3] |
| FSEN-43 | 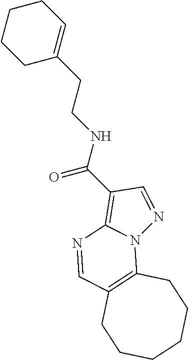 [SMILES: O=C(NCCC1=CCCCC1)c1cnn2c3c(cnc12)CCCCCC3] |
| FSEN-44 | 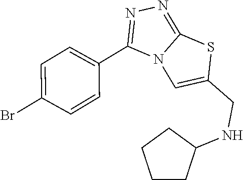 [SMILES: Brc1ccc(-c2nnc3sc(CNC4CCCC4)cn23)cc1] |
| FSEN-45 | 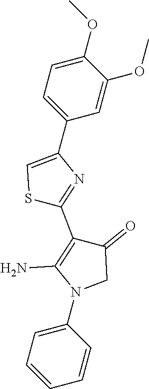 [SMILES: COc1ccc(-c2csc(C3=C(N)N(c4ccccc4)CC3=O)n2)cc1OC] |
| FSEN-46 | 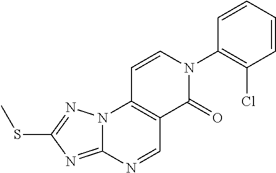 [SMILES: CSc1nc2ncc3c(=O)n(-c4ccccc4Cl)ccc3n2n1] |

In some cases, the FSP1 inhibitor from Table A is a compound having a structure of:

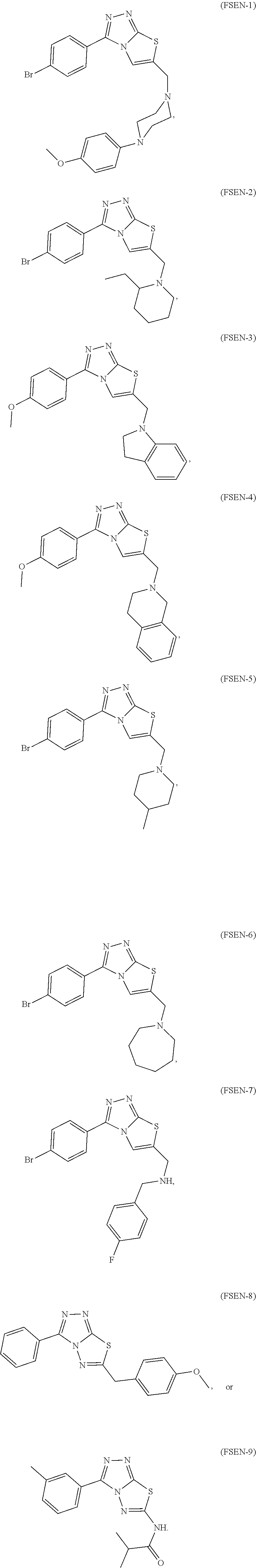

The FSP1 inhibitors disclosed herein can be used in methods of inhibiting FSP1 in a cell. In these methods, a cell is contacted with a disclosed FSP1 inhibitor, or pharmaceutical composition thereof, in an amount effective to inhibit FSP1. In various cases, the contacting includes administering the compound or pharmaceutical composition to a subject.

The term “pharmaceutically acceptable salt” refers to the relatively non-toxic, inorganic and organic acid addition salts of a compound provided herein. These salts can be prepared in situ during the final isolation and purification of a compound provided herein, or by separately reacting the compound in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66:1-19.)

In some embodiments, a compound provided herein may contain one or more acidic functional groups and, thus, is capable of forming pharmaceutically acceptable salts with pharmaceutically acceptable bases. The term “pharmaceutically acceptable salts” in these instances refers to the relatively non-toxic inorganic and organic base addition salts of a compound provided herein. These salts can likewise be prepared in situ during the final isolation and purification of the compound, or by separately reacting the purified compound in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

### Cancers for Treatment

The FSP1 inhibitors disclosed herein can be used in methods for treating various cancers in a subject suffering therefrom. In some cases, the cancer expresses FSP1, e.g., the cancer has a detectable level of FSP1. In some cases, the cancer is GPX4-inhibitor resistant. In some cases, the cancer is ferroptosis-resistant.

In some cases, the subject has been previously exposed to a cancer therapy (i.e., the FSP1 inhibitor is a second or higher line therapy). In some cases, the prior therapy was one that triggers oxidative lipid damage (e.g., photodynamic therapy or radiation). In some cases, the prior therapy was a GPX4 inhibitor. In some cases, the prior therapy was a glutathione synthesis inhibitor. In some cases, the prior therapy was a system xc-inhibitor. In some cases, the prior therapy was an ubiquinone synthesis pathway inhibitor.

Specifically contemplated cancers for treatment include, but are not limited to lung cancer, liver cancer, glial cancer, bone cancer, connective tissue cancer, pancreatic cancer, neuroblastoma, colorectal cancer, astrocytoma, adenocarcinoma, breast cancer, brain cancer, liver cancer, kidney cancer, skin cancer, and intestinal cancer.

### Combination Therapy

FSP1 inhibition alone was not sufficient to trigger ferroptosis in most of the cell types under the culture conditions examined here, except for the A375 melanoma cells. It is worth noting that FSP1 KO dramatically slowed tumor growth in an in vivo xenograft model of KEAP1 deficient lung cancer, indicating that FSP1 inhibition is sufficient to induce ferroptosis under some in vivo conditions. This may reflect the unique contribution of the in vivo tumor microenvironment and higher levels of PUFAs and ROS. Importantly, the observation that FSP1 KO impairs tumor growth in vivo raises the possibility that small molecule FSP1 inhibition may be effective as a monotherapy for specific cancers. The result also highlights the importance of future studies to explore FSP1 inhibition in in vivo cancer models.

The disclosures herein are consistent with the utility of FSP1 inhibition in combinatorial therapeutic regimes together with inhibitors of the GSH-GPX4 pathway. It is likely that FSP1 inhibition will sensitize cancer cells to standard of care treatments that trigger ROS generation and ferroptosis such as radiotherapy, photodynamic therapy, and immunotherapy. Indeed, FSP1 KO and iFSP1 treatment sensitize cancer cells to radiotherapy.

Thus, also provided herein are methods of treating cancer in a subject suffering therefrom by administering a FSP1 inhibitor as disclosed herein in combination with a second therapeutic agent or regimen. Specifically contemplated additional therapeutic agents include, but are not limited to one or more of the following ferroptosis inducers including a GPX4 inhibitor, a system xc-inhibitor, an inhibitor of glutathione synthesis, or a endoperoxide. Specifically contemplated additional therapeutic regimen include, but are not limited to one or more of radiation, and photodynamic therapy. Some GPX4 inhibitors are disclosed in Eaton, J. K. et. al. JACS 141 (51), 20407-20415 (2019), Eaton, J. K. et. al. Biorg Med Chem Lett. 30 (23) 127538 (2020), and Stockwell, B. R. et al. Cell Chem. Biol. 27 (4) 365-375 (2020) each of which is incorporated by reference in its entirety. Some specifically contemplated GPX4 inhibitors include, but are not limited to, one or more of DPI3, DPI4, DPI6, DPI7, DPI8, DPI9, DPI10, DPI12, DPI13, DPI15, DPI17, DPI18, DPI19, FIN56, JKE-1674, JKE-1716, ML162, ML210, RSL3, FINO2, Altretamine, NSC144988, and Withaferin A. Specifically contemplated xc-inhibitors include but are not limited to erastin, erastin2, imidazole ketone erastin, piperazine erastin, DPI2, RSL5, glutamate, sulfasalazine, and sorafenib. Specifically contemplated glutathione synthesis inhibitors include, but are not limited to buthionine sulfoximine and cyst (e) inase. Specifically contemplated endoperoxides include artemisinin, dihydroartemisinin (DHA), artemether, arteether, artesunate, artelininic acid, artesunate, artelinate, artemisone, 3-artesanilide, artefenomel, FINO2, and FINO3. Other endoperoxides are disclosed in, e.g., WO 2007/125397, WO 2004/041176, and WO 2014/011120, each of which is incorporated by reference in its entirety.

As shown in the below examples, FSP1 inhibition synergizes with the endoperoxide DHA to induce ferroptosis. DHA is a commonly used antimalarial that has been explored as an anticancer drug, and several recent studies indicate that DHA is capable of triggering ferroptosis types. The endoperoxide bridge within DHA reacts with and oxidizes iron, leading to the production of reactive oxygen species and lipid peroxides. In H460 lung cancer cells, DHA induced minimal amounts of ferroptosis on its own, but its ability to induce ferroptosis was greatly enhanced by co-treatment with FSEN1. It remains possible that DHA operates through multiple mechanisms, oxidizing iron and triggering lipid peroxide formation and indirectly inhibiting GPX4 similar to what was reported for the endoperoxide ferroptosis inducer FINO2 (see, e.g., Gaschler, et al. (2018). Nat Chem Biol 14, 507-515). These findings highlight the potential of endoperoxide containing compounds (e.g., FINO2, FINO3, DHA) as therapeutic agents to induce ferroptosis by increasing iron oxidation, lipid peroxidation, and potentially GPX4 inhibition.

### Pharmaceutical Compositions

Also included are the pharmaceutical compositions themselves. Pharmaceutical compositions typically include a pharmaceutically acceptable carrier. Thus, provided herein are pharmaceutical compositions that include a compound described herein (e.g., FSEN1-9, or a pharmaceutically acceptable salt thereof), as previously described herein, and one or more pharmaceutically acceptable carriers.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent or encapsulating material. As used herein the language “pharmaceutically acceptable carrier” includes buffer, sterile water for injection, solvents, dispersion media, coatings, antibacterial and antifungal agents, isotonic and absorption delaying agents, and the like, compatible with pharmaceutical administration. Each carrier must be “acceptable” in the sense of being compatible with the other ingredients of the composition and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted β-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions. In certain embodiments, pharmaceutical compositions provided herein are non-pyrogenic, i.e., do not induce significant temperature elevations when administered to a patient.

The term “pharmaceutically acceptable salt” refers to the relatively non-toxic, inorganic and organic acid addition salts of a compound provided herein. These salts can be prepared in situ during the final isolation and purification of a compound provided herein, or by separately reacting the compound in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66:1-19.)

In some embodiments, a compound provided herein may contain one or more acidic functional groups and, thus, is capable of forming pharmaceutically acceptable salts with pharmaceutically acceptable bases. The term “pharmaceutically acceptable salts” in these instances refers to the relatively non-toxic inorganic and organic base addition salts of a compound provided herein. These salts can likewise be prepared in situ during the final isolation and purification of the compound, or by separately reacting the purified compound in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

A pharmaceutical composition may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars and the like into the compositions. In addition, prolonged absorption of the injectable pharmaceutical form may be brought about by the inclusion of agents which delay absorption such as aluminum monostearate and gelatin.

Compositions prepared as described herein can be administered in various forms, depending on the disorder to be treated and the age, condition, and body weight of the patient, as is well known in the art. For example, where the compositions are to be administered orally, they may be formulated as tablets, capsules, granules, powders, or syrups; or for parenteral administration, they may be formulated as injections (intravenous, intramuscular, or subcutaneous), drop infusion preparations, or suppositories. For application by the ophthalmic mucous membrane route, they may be formulated as eye drops or eye ointments. These compositions can be prepared by conventional means in conjunction with the methods described herein, and, if desired, the active ingredient may be mixed with any conventional additive or excipient, such as a binder, a disintegrating agent, a lubricant, a corrigent, a solubilizing agent, a suspension aid, an emulsifying agent, or a coating agent.

Compositions suitable for oral administration may be in the form of capsules (e.g., gelatin capsules), cachets, pills, tablets, lozenges (using a flavored basis, usually sucrose and acacia or tragacanth), powders, troches, granules, or as a solution or a suspension in an aqueous or non-aqueous liquid, or as an oil-in-water or water-in-oil liquid emulsion, or as an elixir or syrup, or as pastilles (using an inert matrix, such as gelatin and glycerin, or sucrose and acacia) and/or as mouthwashes, and the like, each containing a predetermined amount of a compound provided herein as an active ingredient. A composition may also be administered as a bolus, electuary, or paste. Oral compositions generally include an inert diluent or an edible carrier.

Pharmaceutically compatible binding agents, and/or adjuvant materials can be included as part of an oral composition. In solid dosage forms for oral administration (capsules, tablets, pills, dragees, powders, granules, and the like), the active ingredient can be mixed with one or more pharmaceutically acceptable carriers, such as sodium citrate or dicalcium phosphate, and/or any of the following: (1) fillers or extenders, such as starches, cyclodextrins, lactose, sucrose, saccharin, glucose, mannitol, and/or silicic acid; (2) binders, such as, for example, carboxymethylcellulose, microcrystalline cellulose, gum tragacanth, alginates, gelatin, polyvinyl pyrrolidone, sucrose, and/or acacia; (3) humectants, such as glycerol; (4) disintegrating agents, such as agar-agar, calcium carbonate, potato, corn, or tapioca starch, alginic acid, Primogel, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, Sterotes, solid polyethylene glycols, sodium lauryl sulfate, and mixtures thereof; (10) a glidant, such as colloidal silicon dioxide; (11) coloring agents; and (12) a flavoring agent such as peppermint, methyl salicylate, or orange flavoring. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of a powdered compound moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, microspheres, and/or nanoparticles. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active compound(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions suitable for parenteral administration can include one or more compounds provided herein in combination with one or more pharmaceutically acceptable sterile aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the composition isotonic with the blood of the intended recipient or suspending or thickening agents.

Examples of suitable aqueous and nonaqueous carriers which may be employed in the pharmaceutical compositions provided herein include water for injection (e.g., sterile water for injection), bacteriostatic water, ethanol, polyols (such as glycerol, propylene glycol, polyethylene glycol such as liquid polyethylene glycol, and the like), sterile buffer (such as citrate buffer), and suitable mixtures thereof, vegetable oils, such as olive oil, injectable organic esters, such as ethyl oleate, and Cremophor EL™ (BASF, Parsippany, NJ). In all cases, the composition must be sterile and should be fluid to the extent that easy syringability exists. Proper fluidity can be maintained, for example, by the use of coating materials, such as lecithin, by the maintenance of the required particle size in the case of dispersions, and by the use of surfactants.

The composition should be stable under the conditions of manufacture and storage and must be preserved against the contaminating action of microorganisms such as bacteria and fungi. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the methods of preparation are freeze-drying (lyophilization), which yields a powder of the active ingredient plus any additional desired ingredient from a previously sterile-filtered solution thereof.

Injectable depot forms can be made by forming microencapsule or nanoencapsule matrices of a compound provided herein in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly (anhydrides). Depot injectable compositions are also prepared by entrapping the drug in liposomes, microemulsions or nanoemulsions, which are compatible with body tissue.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798. Additionally, intranasal delivery can be accomplished, as described in, inter alia, Hamajima et al., Clin. Immunol. Immunopathol., 88 (2), 205-10 (1998). Liposomes (e.g., as described in U.S. Pat. No. 6,472,375, which is incorporated herein by reference in its entirety), microencapsulation and nanoencapsulation can also be used. Biodegradable targetable microparticle delivery systems or biodegradable targetable nanoparticle delivery systems can also be used (e.g., as described in U.S. Pat. No. 6,471,996, which is incorporated herein by reference in its entirety).

Systemic administration of a therapeutic compound as described herein can also be by transmucosal or transdermal means. Dosage forms for the topical or transdermal administration of a compound provided herein include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required. For transmucosal or transdermal administration, penetrants appropriate to the barrier to be permeated are used in the composition. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The ointments, pastes, creams, and gels may contain, in addition to one or more compounds provided herein, excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to a compound provided herein, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

A compound provided herein can be administered by aerosol. This is accomplished by preparing an aqueous aerosol, liposomal preparation, or solid particles containing a compound or composition provided herein. A nonaqueous (e.g., fluorocarbon propellant) suspension could be used. In some embodiments, sonic nebulizers are used because they minimize exposing the agent to shear, which can result in degradation of the compound.

Ordinarily, an aqueous aerosol can be made by formulating an aqueous solution or suspension of the agent together with conventional pharmaceutically acceptable carriers and stabilizers. The carriers and stabilizers vary with the requirements of the particular composition, but typically include nonionic surfactants (TWEEN® (polysorbates), PLURONIC® (poloxamers), sorbitan esters, lecithin, CREMOPHOR® (polyethoxylates)), pharmaceutically acceptable co-solvents such as polyethylene glycol, innocuous proteins like serum albumin, sorbitan esters, oleic acid, lecithin, amino acids such as glycine, buffers, salts, sugars, or sugar alcohols. Aerosols generally are prepared from isotonic solutions.

Transdermal patches have the added advantage of providing controlled delivery of a compound provided herein to the body. Such dosage forms can be made by dissolving or dispersing the agent in the proper medium. Absorption enhancers can also be used to increase the flux of the compound across the skin. The rate of such flux can be controlled by either providing a rate controlling membrane or dispersing the compound in a polymer matrix or gel.

The pharmaceutical compositions can also be prepared in the form of suppositories or retention enemas for rectal and/or vaginal delivery. Compositions presented as a suppository can be prepared by mixing one or more compounds provided herein with one or more suitable nonirritating excipients or carriers comprising, for example, cocoa butter, glycerides, polyethylene glycol, a suppository wax or a salicylate, which is solid at room temperature, but liquid at body temperature and, therefore, will melt in the rectum or vaginal cavity and release the active agent. Compositions which are suitable for vaginal administration also include pessaries, tampons, creams, gels, pastes, foams, or spray compositions containing such carriers as are known in the art to be appropriate.

The therapeutic compounds may also be prepared with carriers that will protect the therapeutic compounds against rapid elimination from the body, such as a controlled release composition, including implants and microencapsulated delivery systems. Biodegradable, biocompatible polymers can be used, such as ethylene vinyl acetate, polyanhydrides, polyglycolic acid, collagen, polyorthoesters, and polylactic acid. Such compositions can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811, which is incorporated herein by reference in its entirety.

A compound provided herein may be administered to humans and other animals for therapy by any suitable route of administration, including orally, nasally, as by, for example, a spray, rectally, intravaginally, parenterally, intracistemally, and topically, as by powders, ointments or drops, including buccally and sublingually. Regardless of the route of administration selected, a compound provided herein, which may be used in a suitable hydrated form, and/or the pharmaceutical compositions provided herein, is formulated into a pharmaceutically acceptable dosage form by conventional methods known to those of skill in the art. In another embodiment, the pharmaceutical composition is an oral solution or a parenteral solution. Another embodiment is a freeze-dried preparation that can be reconstituted prior to administration. As a solid, this composition may also include tablets, capsules or powders.

Actual dosage levels of the active ingredients in the pharmaceutical compositions provided herein may be varied so as to obtain “therapeutically effective amount,” which is an amount of the active ingredient effective to achieve the desired therapeutic response for a particular patient, composition, and mode of administration, without being toxic to the patient.

The concentration of a compound provided herein in a pharmaceutically acceptable mixture will vary depending on several factors, including the dosage of the compound to be administered, the pharmacokinetic characteristics of the compound(s) employed, and the route of administration. In some embodiments, the compositions provided herein can be provided in an aqueous solution containing about 0.1-10% w/v of a compound disclosed herein, among other substances, for parenteral administration. Typical dose ranges can include from about 0.01 to about 50 mg/kg of body weight per day, given in 1-4 divided doses. Each divided dose may contain the same or different compounds. The dosage will be a therapeutically effective amount depending on several factors including the overall health of a patient, and the composition and route of administration of the selected compound(s).

Dosage forms or compositions containing a compound as described herein in the range of 0.005% to 100% with the balance made up from non-toxic carrier may be prepared. Methods for preparation of these compositions are known to those skilled in the art. The contemplated compositions may contain 0.001%-100% active ingredient, in one embodiment 0.1-95%, in another embodiment 75-85%. Although the dosage will vary depending on the symptoms, age and body weight of the patient, the nature and severity of the disorder to be treated or prevented, the route of administration and the form of the drug, in general, a daily dosage of from 0.01 to 2000 mg of the compound is recommended for an adult human patient, and this may be administered in a single dose or in divided doses. The amount of active ingredient which can be combined with a carrier material to produce a single dosage form will generally be that amount of the compound which produces a therapeutic effect.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

It is to be understood that while the disclosure is read in conjunction with the detailed description thereof, the foregoing description is intended to illustrate and not limit the scope of the disclosure, which is defined by the scope of the appended claims. Other aspects, advantages, and modifications are within the scope of the following claims.

### Examples

### Experimental Procedures

### Cell Lines and Culture Conditions

A549^{N}, HCC1143^{C}, NCI-H460^{C}, NCI-H460^{C} GPX4^{KO} and FSP1^{KO} lines were cultured in RPMI1640 with l-glutamine (corning). HT-1080^{N}, U-2OS^{N}, T98G^{N}, Huh7^{C}, and A375 cells were cultured in DMEM with I-glutamine and without sodium pyruvate (Corning). 501MEL and SKMEL28 cells were cultured in DMEM high glucose with I-glutamine and without sodium pyruvate (Corning). Cells (HT-1080^{N}, U-2OS^{N}, T98G^{N}) were generated through stable expression of nuclear-localized mKate2 (denoted by an additional superscript N′). All media were supplemented with 10% fetal bovine serum (FBS, Thermo Fisher Scientific and Gemini Bio Products), and all cell lines were grown at 37° C. with 5% CO₂. All cell lines were tested for mycoplasma and were not authenticated.

### Generation of CRISPR-Cas9 Genome-Edited Cell Lines

NCI-H460^{C} FSP1^{KO} lines were generated by infection with lentiCRISPR v2-Blast (Addgene plasmid no. 83489) virus, and NCI-H460^{C} GPX4KO lines were generated by infection with lentiCRISPR v2-Hygro (Addgene plasmid no. 98291) virus, as previously described (Jiang, et al. (2021). Ferroptosis: mechanisms, biology and role in disease. Nat Rev Mol Cell Biol 22, 266-282). Cells expressing sgSAFE-mCherry (denoted by an additional superscript ‘C’) were generated from the respected parental cells via transduction with the lentiviral sgRNA expression vector with mCherry pMCB320 (Addgene plasmid no. 89359) virus, which directs the expression of cytosolic sgSAFE-mCherry. Polyclonal sgSAFE-mCherry expressing cells were selected for using puromycin or hygromycin respectively. H460C polyclonal pools were selected puromycin and further enriched using FACS (UC Berkeley shared FACS Facility).

### Plasmids

FSP1 inserted into the pET-His6-TEV vector was previously described (Bersuker et. al. 2019). For protein expression of NQO1, NQO1 (WT) lacking the ATG start codon was generated by PCR amplification of NQO1 from an NQO1-GFP pcDNA5/FRT/TO plasmid generated as disclosed in Bersuker, K. et al. Nature 575, 688-692 (2019). The NQO1 (WT) amplicon was inserted into the pET-His6-TEV vector (Addgene plasmid no. 29653), C-terminal to the His6-TEV tag using restriction enzyme-independent fragment insertion by polymerase incomplete primer extension.

### Chemicals and Reagents

Reagents used in this study include: RSL3 (Cayman Chemical), Fer-1 (Cayman Chemical), idebenone (Cayman Chemical), DFO (Cayman Chemical), ML162 (Cayman Chemical), ZVAD (OMe)-FMK (Cayman Chemical), necrostatin-1 (Cayman Chemical), DHA (Selleck Chemical), puromycin (Thermo Fisher Scientific), SYTOX Green Dead Cell Stain (Thermo Fisher Scientific), polybrene (Sigma-Aldrich), coenzyme Q1 (Sigma-Aldrich), resazurin (Thermo Fisher Scientific) and NADH (Sigma-Aldrich). A 100,000-member compound Diverse Library and a 15,000 compound Antibacterial Library were obtained from ChemDiv. The 1,200 compound FDA approved, and 4,170 compounds Bioactive libraries were obtained from TargetMol. Compounds were stamped into 384 well Non-Binding Surface (NBS) plates (Corning) using a Cybio Well Vario liquid handler (Analytik-Jena, Germany).

### Small Molecule Screen for FSP1 Inhibitors Using In Vitro Activity and Cell-Based Assays

For small molecule drug screen, 0.5 μL of compounds were stamped into 384 well NBS plates (Corning) in dose response diluted 2-fold with a high of 2 mM. 60 mL of purified His tagged FSP1 (WT) protein at 50 nM was prepared and 12.5 μL of this protein solution was aliquoted into 384 well NBS-plates (Corning) containing compound and allowed to incubate for 30 min at room temperature. After 30 min incubation 12.5 μL of reaction buffer (1 mM NADH, 800 UM CoQ1) was added into the 384 well NBS-plates containing protein and compounds for a final concentration of 25 nM His tagged FSP1 (WT) protein, 500 UM NADH (Sigma-Aldrich) and 400 UM CoQ1 (Sigma-Aldrich). Each well contained a 25 μL mixture and 40 UM compound in the primary screen. All aliquots for in-vitro drug screen were completed with an Analytik-Jena Cybio Well Vario liquid handler. 2 mM compounds were stored in 100% DMSO in 384 well plates. Wells were homogenized with a Bioshake 3000 ELM orbital shaker at 2,400 rpm for 45 s and condensates were allowed to settle for 30 min before scanning. Measurements were taken at 355 nm with a EnVision 2104 multilabel plate reader (PerkinElmer). No protein data control was used for background subtraction prior to upload to CCDvault for normalization to DMSO vehicle control wells. Compounds with confirmed data that have normalized absorbance values of less than 0.164 were chosen for dose response. Wells that exhibit 3 standard deviations from the untreated sample and a no protein control were selected for dose-response screening. The same His-tagged FSP1 (WT) protein were tested against candidate drugs using a 10-point serial dilution starting at 40 μM using the same procedure.

For cell-based screen, cells were seeded in triplicate at a density of 500-750 cells per 50 μL per well in black 384-well plates (NUNC) and (Corning) 24 hr before start of imaging. After 24 hr, an additional 50 μL of drug infused medium containing 30 nM SYTOX Green Dead Cell Stain was carefully placed into the wells on top of existing medium. The plates were immediately transferred to an IncuCyte S3 imaging system (Essen Bioscience) enclosed in an incubator set to 37° C. and 5% CO₂. One image per well were captured in the phase, green, and red channels every 1.5 or 3 hr over a 24 hr period, and the ratio of SYTOX Green-positive objects (dead cells) to SYTOX Green-positive plus Sg-SAFE mCherry-positive objects (total cells) was quantified using S3 image analysis software (Essen Bioscience). For each treatment condition, the SYTOX-to-′SYTOX+mCherry′-object ratio was plotted against the 24 hr imaging interval, the Area Under the Curve (AUC) was calculated and the average AUC was plotted using Prism (GraphPad). To calculate the half-maximal effective concentration (EC50) values, the AUC curve was fit to a variable slope function comparing response to drug concentration.

Lipid peroxidation assay: Cells seeded in a 6-well plate were treated with 200 nM RSL3 for 5 hr and washed once with DPBS containing calcium and magnesium. Cells then were incubated in DPBS containing 5 UM BODIPY 581/591 C11 (Invitrogen, #D3861) at 37° C. for 10 min and washed 3× with DPBS without calcium or magnesium. Cells were detached from the plate with trypsin, and green fluorescence was analyzed by flow cytometry (>10,000 cells) on a BD LSRFortessa. Data were analyzed using FlowJo.

Spheroid/3D cell culture: 2500 NCI-H460 cells with 100 μL full serum RPMI media were seeded in 96-well Black/Clear Round Bottom Ultra-Low Attachment Spheroid Microplate (Corning, #4515). Cells were incubated at 37° C. for 30 min before another 100 μL RPMI media containing 2% Matrigel (Corning #354234) was added. Plates were centrifuged at 750×g for 15 min and grew at 37° C. for 2 days. For FSEN1 and RSL3 treatment, 100 μL RPMI media was slowly removed without disturbing the spheroid. Another 100 μL RPMI media containing 1% Matrigel, 60 nM SYTOX Green dye, 10 UM FSEN1 or 10 μM RSL3 or both was added back into each well. Spheroids were imaged in IncuCyte S3 with a 10× objective.

FDA and Bioactive Library Screen: For FDA and Bioactive Library Screen experiments, cells were seeded at a density of 750 cells per 25 uL per well in black 384-well plates (NUNC) and (Corning) 24 hr before start of imaging. After 24 hr, an additional 25 μL of drug infused medium containing 30 nM SYTOX Green Dead Cell Stain was carefully placed into the wells on top of existing medium. The plates were immediately transferred to an IncuCyte S3 imaging system (Essen Bioscience) enclosed in an incubator set to 37° C. and 5% CO₂. One image per well were captured in the phase, green, and red channels every 4 hr over a 24 hr period, and the ratio of SYTOX Green-positive objects (dead cells) to SYTOX Green-positive plus SgSAFE mCherry-positive objects (total cells) was quantified using S3 image analysis software (Essen Bioscience). For each treatment condition, the SYTOX-to-‘SYTOX+mCherry’-object ratio was plotted against the 24 hr imaging interval, the Area Under the Curve (AUC) was calculated, and the average AUC was plotted as a function of drug concentration (for example, RSL3) using Prism (GraphPad). To calculate the half-maximal effective concentration (EC50) values, the AUC curve was fit to a variable slope function comparing response to drug concentration.

Synergy experiments: For cell-based synergy experiments, cells were seeded at a density of 750 cells per 25 μL per well in four black 384-well plates (Corning) 24 hr before start of imaging. After 24 hr, an additional 25 μL of drug infused medium containing 30 nM SYTOX Green Dead Cell Stain was carefully placed into the wells on top of existing medium. The plates were immediately transferred to an IncuCyte S3 imaging system (Essen Bioscience) enclosed in an incubator set to 37° C. and 5% CO₂. One image per well were captured in the phase, green, and red channels every 4 hr over a 24 hr period, and the ratio of SYTOX Green-positive objects (dead cells) to SYTOX Green-positive plus Sg-SAFE mCherry-positive objects (total cells) was quantified using S3 image analysis software (Essen Bioscience). For each treatment condition, the SYTOX-to-′SYTOX+mCherry′-object ratio was plotted against the 24 hr imaging interval, the Area Under the Curve (AUC) was calculated, and the average AUC was plotted as a function of drug concentration (for example, RSL3) using Prism (GraphPad). To calculate the half-maximal effective concentration (EC50) values, the AUC curve was fit to a variable slope function comparing response to drug concentration.

Compound preparation for synergy experiments: Vehicle, RSL3 and DHA combinations were stamped in grid format 1:1 at 4×concentrations. The compounds were then diluted with SYTOX green infused media containing either vehicle, 0.10 μM, 1 μM, or 10 μM FSEN1 to the appropriate 2×concentration (PlateOne, #1884-2410). Compounds were then homogenized with a Bioshake 3000 ELM orbital shaker at 2,400 rpm for 45 sec prior to treatment.

### Protein Purification and Activity Assays

Expression vectors were transformed into LOBSTR-BL21 (DE3) competent cells (Kerafast) and LB cultures were inoculated for overnight growth at 37° C. while shaking. The following day, the cultures were diluted 1:100 into 500 ml of LB and allowed to grow to an optical density at 600 nm (OD₆₀₀) of 0.6, at which point the cultures were allowed to incubate at 20° C. The cultures were grown further to an OD₆₀₀ of 0.7 and induced with 1 mM isopropyl β-D-1-thiogalactopyranoside (IPTG) overnight. Bacteria was pelleted by centrifugation and bacterial pellets resuspended in 2 ml of cold lysis buffer containing 50 mM potassium phosphate pH 8.0, 300 mM potassium chloride, and 30 mM imidazole, supplemented with 1×complete, Mini, EDTA-free Protease Inhibitor Cocktail. The resuspended cells were sonicated 5×on ice at 50% power for 15 s, with 2-min incubations on ice in between sonications. Lysate was then centrifuged at 20,000g for 30 min at 4° C. The supernatant was combined with 200 μl of Ni-NTA agarose beads (Thermo Fisher Scientific) washed 3× with lysis buffer, and the supernatant-bead mixture was rotated for 30 min at 4° C. The beads were subsequently washed 5× with cold lysis buffer, and bound proteins were eluted by incubating beads for 15 min in 500 μl of cold lysis buffer containing 250 mM imidazole while rotating. The eluted proteins were concentrated into PBS containing 10% glycerol and ran on a HiLoad 16/600 Superdex 75 pg, concentrated, aliquoted, and snap-frozen in liquid N₂. Protein concentration was determined by measuring the absorbance at 280 nm and using the bicinchoninic acid (BCA) protein assay (Thermo Fisher Scientific).

To measure NADH oxidation kinetics, 25 nM recombinant FSP1 was combined with 500 UM NADH and C400 UM Coenzyme Q1 in a total volume of 100 μl PBS. The change in absorbance at 355 nm, corresponding to NADH oxidation, was determined over the course of 4 h. All measurements were taken using a SpectraMax i3 Multi-Mode Platform plate reader (Molecular Devices).

FSP1 Kinetics (NADH): To measure FSP1 kinetics, FSEN1 was dissolved and diluted in DMSO, and recombinant purified FSP1 was diluted in PBS. 0.5 μL FSEN1 and 12.5 μL FSP1 were mixed and incubated for 30 minutes in a NBS polystyrene 384-well plate (Corning Ref: 3640) at RT. Vehicle control wells included 0.5 μL DMSO and were absent of any FSEN1. NADH (Millipore Cat: 481913) was then dissolved and diluted in PBS and added to the plate wells. CoQ-Coumarin (Cayman Item: 29554) was dissolved in DMSO, diluted in PBS, and added to the plate wells to start the reaction. Final well volume was 25 μL, and final concentrations were 25 nM FSP1, 10UM CoQ-Coumarin, 1-50 μM NADH, and 50-500 nM FSEN1. Following addition of CoQ-Coumarin, the well-volumes were mixed by an orbital shaker for 20 seconds at an amplitude of 5 mm and 120 rpm. Reduced CoQ-Coumarin fluorescence (Ex: 405 nm, Em: 475 nm) was measured as read-out of enzymatic product formation on a kinetic cycle with an interval time of 20 seconds at RT. All data was acquired using a Tecan infinite M1000. Raw data from 3 biological replicates were then plotted and initial slopes were calculated using a linear regression in prism (GraphPad).

FSP1 Kinetics (CoQ-Coumarin): To measure FSP1 kinetics, FSEN1 was dissolved and diluted in DMSO, and recombinant purified FSP1 was diluted in PBS. 0.5 μL FSEN1 and 12.5 μL FSP1 were mixed and incubated for 30 minutes in a NBS polystyrene 384-well plate (Corning Ref: 3640) at RT. Vehicle control wells included 0.5 μL DMSO and were absent of any FSEN1. NADH (Millipore Cat: 481913) was dissolved and diluted in PBS, and CoQ-Coumarin (Cayman Item: 29554) was dissolved in DMSO and diluted in PBS. Diluted NADH and CoQ-Coumarin were combined into a 2×reaction mix and added to the plate wells to start the reaction. Final well volume was 25 μL, and final concentrations were 6.25 nM FSP1, 1-30UM CoQ-Coumarin, 200 μM NADH, and 12.5-125 nM FSEN1. Reduced CoQ-Coumarin fluorescence (Ex: 405 nm, Em: 475 nm) was measured as read-out of enzymatic product formation on a kinetic cycle with an interval time of 15 seconds at RT. All data was acquired using a Tecan Spark. Raw data from 3 biological replicates were then plotted and initial slopes were calculated using a linear regression in prism (GraphPad).

FSP1 Kinetics (CoQ1): To measure FSP1 kinetics, FSEN1 was dissolved and diluted in DMSO, and recombinant purified FSP1 was diluted in PBS. 0.5 μL FSEN1 and 12.5 μL FSP1 were mixed and incubated for 30 minutes in in a NBS polystyrene 384-well plate (Corning Ref: 3640) at RT. Vehicle control wells included 0.5 μL DMSO and were absent of any FSEN1. NADH (Millipore Cat: 481913) was dissolved and diluted in PBS, and CoQ1 (Sigma-Aldrich SKU: C7956) was dissolved in DMSO and diluted in PBS. Diluted NADH and CoQ1 were combined into a 2×reaction mix and added to the plate wells to start the reaction. Final well volume was 25 μL, and final concentrations were 25 nM FSP1, 200-500 μM CoQ1, 500UM NADH, and 0.05-1 μM FSEN1. NADH Absorbance (355 nm) was measured as an inverse read-out of enzymatic product formation on a kinetic cycle with an interval time of 3 minutes at RT. All data was acquired using a Tecan infinite M1000. Raw data from 2 biological replicates were then plotted and initial slopes were calculated using a linear regression in prism (GraphPad).

FSP1 and NQO1% Activity Curves: To measure in vitro activity of FSP1 and NQO1 for IC50 calculation, recombinant purified FSP1 and NQO1 were diluted in PBS. FSEN1 was dissolved and diluted in DMSO, and 0.5 L FSEN1 and 12.5 μL FSP1/NQO1 were mixed on an orbital shaker for 45 seconds at 450 rpm and incubated for 30 minutes in in a NBS polystyrene 384-well plate (Corning Ref: 3640) at RT. NADH (Millipore Cat: 481913) was dissolved and diluted in PBS, and CoQ1 (Sigma-Aldrich SKU: C7956) was dissolved in DMSO and diluted in PBS. Diluted NADH and CoQ1 were combined into a 2×reaction mix and added to the plate wells to start the reaction. Final well volume was 25 μL, and final concentrations were 12.5n M FSP1, 400 UM CoQ1, 500 UM NADH, and 0.01-20 UM FSEN1. NADH Absorbance (355 nm) was measured as an inverse read-out of enzymatic product formation on a kinetic cycle with an interval time of 2.5 minutes at RT. All data was acquired using a Tecan infinite M1000. Raw data from 3 biological replicates were then plotted and initial slopes were calculated using a linear regression in prism (GraphPad), and all rates were normalized to vehicle (0 nM FSEN1) and No Protein controls where the highest and lowest slope values were used as 0 and 100%. Normalized values were then plotted as a function of FSEN1 concentration in log scale, and prism (GraphPad) was used to perform a non-linear regression curve fit for log (inhibitor) vs. normalized (variable) slopes. IC50 values of FSEN1 for FSP1 and NQO1 were obtained from this non-linear regression.

### Western Blotting

Cells were washed two times with PBS prior to lysis in 1% SDS. Samples were then sonicated for 30 sec and incubated for 5 min at 100° C. Protein concentrations were determined using the bicinchoninic acid (BCA) protein assay (Thermo Fisher Scientific), and equal amounts of protein by weight were combined with 1×Laemmli buffer, separated on 4-20% polyacrylamide gradient gels (Bio-Rad Laboratories) and transferred onto nitrocellulose membranes (Bio-Rad Laboratories). Membranes were washed in PBS with 0.1% Tween-20 (PBST) and blocked in PBST containing 5% (w/v) dried milk or 5% (w/v) bovine serum albumin (BSA) for 30 min. Membranes were incubated for 24 h in PBST containing 5% BSA (Akron Biosciences) and primary antibodies. After washing with PBST, membranes were incubated at room temperature for 30 min in 5% BSA and PBST containing fluorescent secondary antibodies. Immunoblots were imaged on a LI-COR imager (LI-COR Biosciences).

The following blotting reagents and antibodies were used: anti-β-actin (Santa Cruz Biotechnology), anti-GPX4 (Abcam), anti-rabbit IRDye800 conjugated secondary (LI-COR Biosciences) and anti-mouse Alexa Fluor 680 conjugated secondary (Invitrogen).

### Results

### Characterization of In Vitro Assays of FSP1 Activity and FSP1-Mediated Ferroptosis Suppression

As a first step toward identifying small molecule inhibitors of FSP1, an in vitro assay of FSP1 activity was characterized that uses a HIS-TEV-FSP1 purified from e. coli, Coenzyme Q1, and NADH. This assay exploits the change in absorbance as NADH is converted to NAD+during the reduction of Coenzyme Q1. As anticipated, the addition of FSP1 results in the oxidation of NADH over time. The FSP1 inhibitor iFSP1 has only been characterized as an FSP1 inhibitor in cells and whether it is a direct FSP1 inhibitor is unknown. Employing an in vitro FSP1 assay, iFSP1 was found to directly inhibit FSP1 Coenzyme Q1 oxidoreductase activity. These data demonstrate that iFSP1 is a direct FSP1 inhibitor and support the ability of this assay to detect direct FSP1 inhibitors. While iFSP1 is a positive control for these studies, the data indicate that iFSP1 is a relatively weak FSP1 inhibitor (IC50=4.009 UM) and emphasize the need for more potent inhibitors.

In addition to the in vitro activity assay, an orthogonal cell-based assay of FSP1 activity was also developed. This assay uses a NCI-H460 (referred to as H460 cells) lung cancer cell line expressing mCherry (H460^{C} C.) in which GPX4 was knocked out using CRISPR-Cas9. The loss of GPX4 results in an exceptional requirement for FSP1 to suppress ferroptosis (Bersuker et. al. 2019). mCherry is used as a live cell marker, which together with SYTOX Green can be used to calculate the fraction of dead cells (i.e., lethal fraction). Indeed, treatment of H460^{C} GPX4KO with iFSP1 is sufficient to trigger ferroptosis in these cells, indicating that this assay can be used as an orthologous assay to characterize the ability of compounds to inhibit FSP1 and induce ferroptosis in cancer cells.

### Chemical Library Screen Identifies Small Molecule Inhibitors of FSP1 Activity

To identify small molecule inhibitors of FSP1, a chemical screen employing an in vitro assay of FSP1 activity was employed. This assay exploits the change in 355 nm absorbance as NADH is oxidized to NAD+during the reduction of CoQ1 by recombinant FSP1. As anticipated, the addition of FSP1 to the reaction mix resulted in a decrease in absorbance over time. Moreover, the decrease in absorbance was blocked in a dose-dependent manner by iFSP1 (IC50 of 4 M). These data demonstrate that iFSP1 is a direct FSP1 inhibitor and validates the activity assay as a method to identify FSP1 inhibitors.

In the primary screen, the effect of 120,370 small molecules on FSP1 activity was analyzed using the in vitro FSP1 activity assay. This screen identified 1,120 candidate FSP1 inhibitors and 660 candidate activators based on a 0.264 normalized absorbance threshold value. Duplicate analyses of the candidate inhibitors and activators were performed for validation. A control lacking FSP1 protein was included to identify small molecules that altered absorbance independently of FSP1. This series of validation steps yielded 323 inhibitors and 75 activators. Finally, triplicate 10-point dose response analyses were performed to determine the in vitro potency of 168 selected of the FSP1 inhibitors. 26 of these compounds have a lower IC50 than iFSP1 (<4 μM) and 11 compounds have an IC50 below 100 nM.

### Small Molecule Inhibitors of FSP1 Trigger Cell Death in a Cancer Cell Model

To determine whether the 168 candidate FSP1 inhibitors are able to inhibit FSP1 in cells, an orthogonal cell-based assay of FSP1 activity was developed. This assay uses NCI-H460^{C} KEAP1 mutant lung cancer cells expressing mCherry (H460^{C} C.) in which GPX4 was knocked out using CRISPR-Cas9 (H460^{C} GPX4^{KO}) or which expressed Cas9 as a control (H460^{C} Cas9). mCherry was used as a live cell marker, which together with the SYTOX Green cell death marker can be used to calculate the fraction of dead cells (i.e., lethal fraction). To validate this assay, H460^{C} Cas9 cells and H460^{C} GPX4^{KO} cells were treated with iFSP1. iFSP1 selectively triggered cell death in H460^{C} GPX4^{KO} cells, but not in the H460^{C} Cas9 cells. These data demonstrate that this assay can be used to characterize the ability of FSP1 inhibitors to inhibit FSP1 and induce ferroptosis in cancer cells.

Employing this assay, the amount of cell death induced by 168 FSP1 inhibitors in the H460^{C} Cas9 and H460^{C} GPX4^{KO} cells was assessed. H460^{C} Cas9 cells were included to identify any small molecules that kill cells through a ferroptosis-independent mechanism. Triplicate 10-point dose response analyses were performed, and cell death was measured using fluorescence time-lapse imaging. ˜50 of the FSP1 inhibitors induced cell death in the H460^{C} GPX4^{KO} cells, but not the H460^{C} Cas9 cells, indicating that these compounds are synthetic lethal with GPX4^{KO} and are not generally toxic to cells.

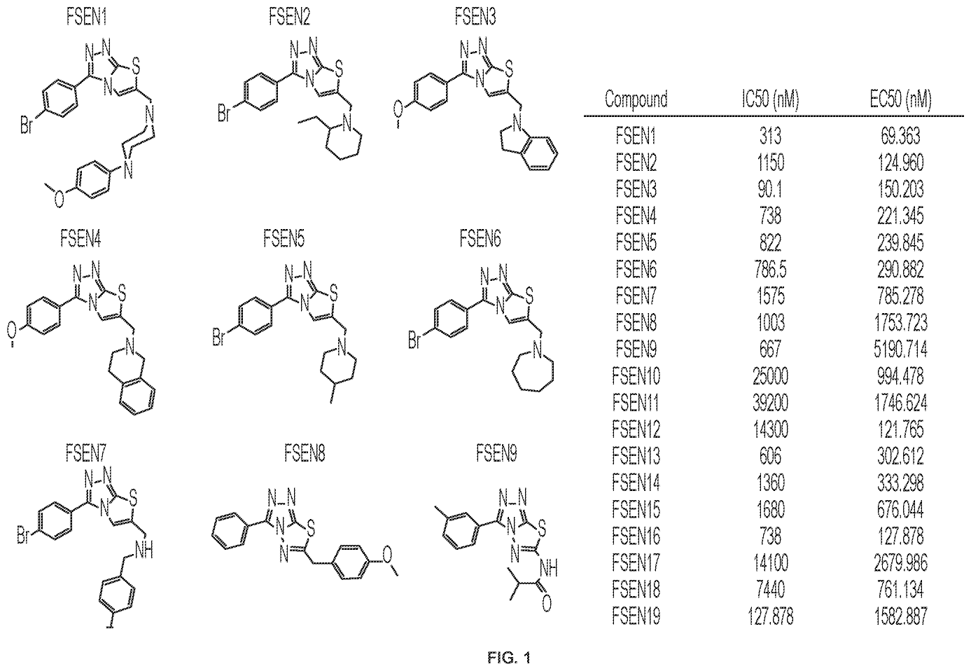

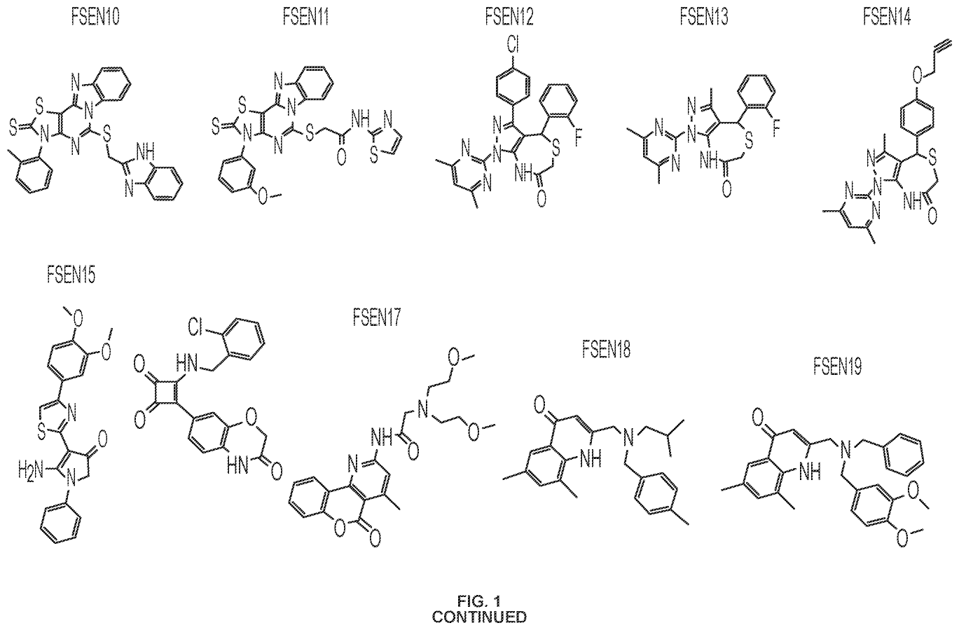

19 of the most potent compounds were tested in 20-point dose response analyses in both the in vitro and cell-based FSP1 assays. As observed in primary and follow up screens, these 19 compounds directly inhibited purified FSP1 in vitro and triggered cell death in the GPX4^{KO} cells, but not in the H460^{C} Cas9 control cells. These validated FSP1 inhibitors are referred to as ferroptosis sensitizer 1-19 (FSEN1-19). The structures of FSEN1-19, their IC50 for inhibition of purified FSP1 activity, and their EC50 for triggering cell death in the H460^{C} GPX4^{KO} cell line are shown in FIG. 1. These compounds can be organized into 7-major groups of structurally related compounds. The largest group (Group 1) contains compounds that share a disubstituted [1,2,4]thiazolo-thiazole ring structure.

FSEN1 is an uncompetitive inhibitor of FSP1 Amongst the newly identified FSP1 inhibitors, FSEN1 exhibited the highest potency in triggering cell death in the H460^{C} C. GPX4^{KO} cells (EC50=69.363 nM). To test the specificity of FSEN1, its ability to inhibit NQO1, another CoQ oxidoreductase that has been implicated in ferroptosis was assessed. In contrast to FSP1, FSEN1 had no effect on the CoQ oxidoreductase activity of NQO1, indicating that FSEN1 does not generally inhibit CoQ oxidoreductases and that FSEN1 exhibits selectivity towards FSP1.

To understand the mechanism of FSP1 inhibition by FSEN1, enzyme kinetics were analyzed using the in vitro FSP1 activity assay in the presence of increasing amounts of its substrates, NADH and a fluorescent ubiquinone analogue CoQ-coumarin. As FSEN1 concentrations increased, a decrease in Kcat values was observed, and the slopes of the Lineweaver-Burke plots were parallel. A similar effect of FSEN1 on the Vmax of FSP1 using CoQ1 as a substrate was observed, but this assay was unable to resolve the Km due to limitations in its sensitivity. These findings reveal that FSEN1 is an uncompetitive inhibitor of FSP1. Thus, FSP1 requires binding to its substrates NADH and CoQ first in order to be permissive for FSEN1 binding, which then yields the inactive complex.

### FSEN1 Triggers Ferroptosis in Cancer Cells by Inhibiting FSP1

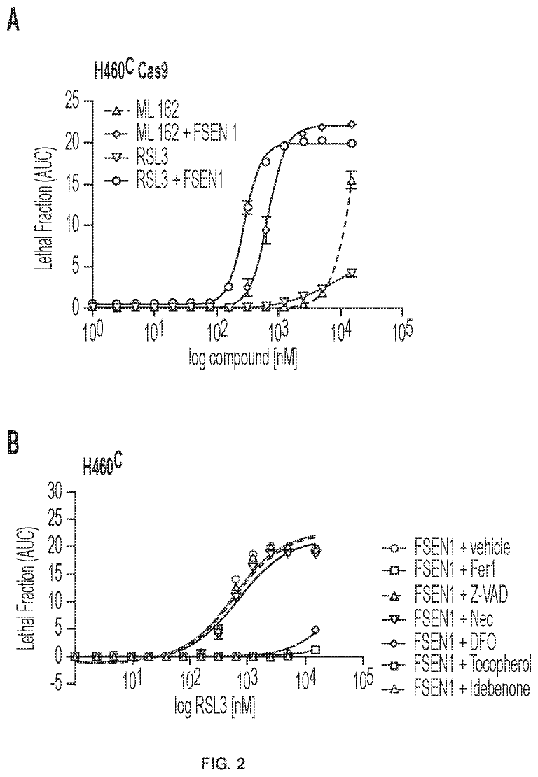

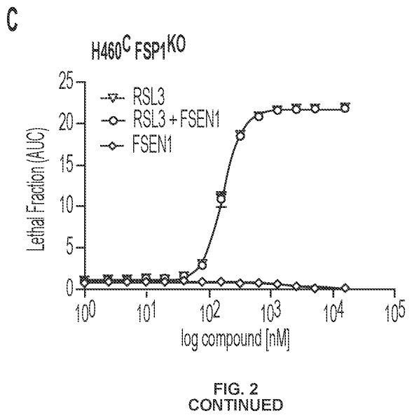

FSEN1 exhibited the highest potency in triggering cell death in the H460^{C} GPX4KO cells (FIG. 1). To further characterize the cell death induced by FSEN1 treatment, its effects on H460^{C} lung cancer cells was examined. Treatment with FSEN1 sensitized H460^{C} cells to two different GPX4 inhibitors, RSL3 and ML162 (FIG. 2A,C). Consistent with FSEN1 sensitizing these cells to ferroptosis, the cell death induced by the co-treatment of FSEN1 with RSL3 was blocked by incubation with known ferroptosis inhibitors, including the radical trapping antioxidants idebenone, ferrostatin-1, and tocopherol, and the iron chelator deferoxamine (DFO) (FIG. 2A-C). In contrast, the apoptosis inhibitor Z-VAD and necroptosis inhibitor Nects had no effect (FIG. 2B).

To determine if FSEN1 sensitizes cells to ferroptosis by inhibiting FSP1, the effect of FSEN1 on RSL3-induced ferroptosis in a H460^{C} cell line was examined in which FSP1 was knocked out using CRISPR-Cas9 (H460^{C} FSP1KO cells). Consistent with previous reports (Bersuker et. al. 2019), H460^{C} FSP1KO cells were greatly sensitized to RSL3-induced cell death (FIG. 2C). Importantly, although FSEN1 sensitized H460^{C} control cells to RSL3-induced cell death, treatment of the H460^{C} FSP1KO cells with FSEN1 did not result in any additional sensitization to RSL3-induced cell death (FIG. 2C). These findings demonstrate that FSEN1 sensitizes H460 lung cancer cells to ferroptosis by inhibiting FSP1 function and not through inhibition of other ferroptosis resistance factors, including other CoQ oxidoreductases implicated in ferroptosis such as NQO1.

High cell densities and cell-cell interactions promote ferroptosis resistance by inducing multiple signaling pathways, including the NF2-YAP and TAZ-EMP1-NOX4 pathways, underscoring the importance of testing ferroptosis sensitivity in 3-dimensional (3-D) tumor models. Spheroids are 3-D aggregates of cancer cells that more closely reflect key characteristics of solid tumor biology, including cell-cell interactions, hypoxia, drug penetration, and interactions with deposited extracellular matrix. Importantly, similar to the 2-D culture experiments, RSL3 and FSEN1 synergized to trigger cell death in H460 cells grown in the 3-D spheroids. Together, these findings indicate that FSEN1 sensitizes H460 lung cancer cells to ferroptosis in both 2-D and 3-D culture models.

### FSEN1 Sensitizes Multiple Cancer Cell Lines of Different Origins to Ferroptosis

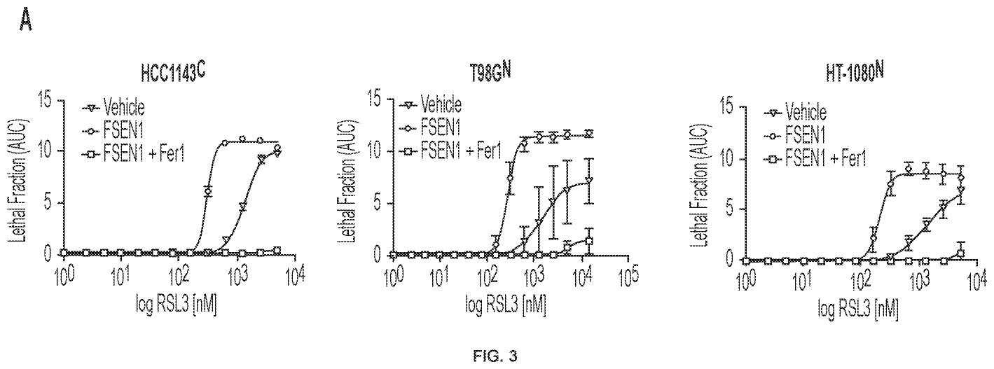

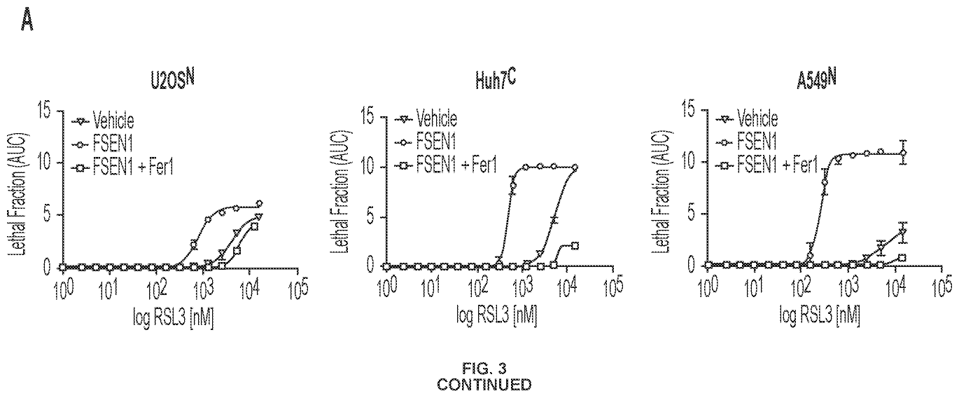

To examine the role of FSP1 in suppressing ferroptosis in different types of cancer, the impact of FSEN1 on RSL3-induced cell death in a panel of cancer cell lines of various tissue origins, including lung (A549N), breast (HCC11439), liver (Huh7° C.), glial (T98G^{N}), bone (U 2-OS^{N}), connective (HT-1080N), lymphoid (RL, SUDHL5), and skin (A375, SKMEL28, 501-MEL) was measured (FIG. 3A,B). FSEN1 sensitized all cancer cells to RSL3-induced ferroptosis to varying extents, and in all cases the cell death was rescued by co-treatment with Fer-1. These findings indicate that FSEN1 can sensitize multiple cancer cell lines of different origins to ferroptosis induced by GPX4 inhibition. Notably, FSEN1 had a particularly large sensitizing effect on ferroptosis induction in A549 lung cancer cells (FIG. 3A). A549, and the H460 cells used in the initial screens, are both lung cancer cell lines with KEAP1 mutations, which results in NRF2-dependent upregulation of FSP1. The prominent role of FSP1 in protecting A549 and H460 cells from ferroptosis correlated with high FSP1 protein levels and low GPX4 levels relative to other cancer cell lines. It was also notable that FSEN1 induced a small amount of ferroptosis in A375 melanoma cells in the absence of an RSL3 co-treatment (FIG. 3B), indicating that A375 cells have a particularly strong dependence on FSP1 for ferroptosis suppression. The amount of sensitization imbued by FSEN1 likely depends on the expression levels of FSP1 and ferroptosis related factors, such as GPX4, DHODH, GCH1, ACSL3, ACSL4, and others.

### FSP1 and Dihydroartemisinin Treatment Synergize to Trigger Ferroptosis in Cancer Cells

As ferroptosis suppression is mediated by several different pathways, it is perhaps not surprising that FSP1 inhibition alone is not sufficient to induce ferroptosis in most cancer cell lines. Furthermore, while FSEN1 is synthetically lethal with GPX4 inhibitors such as RSL3, the in vivo efficacy of RSL3 is known to be limited due to low solubility and poor²⁸. Therefore, a synthetic lethal screen was conducted to identify compounds that eliminate cancer cells specifically in presence of FSEN1. H460^{C} FSP1KO cells were treated for 24 h with a library of 5,370 compounds that includes 1,200 FDA approved drugs and 4,170 bioactive compounds, and lethal fraction was quantified by time-lapse fluorescence imaging. RSL3 was included as a positive control. As expected, RSL3 was cytotoxic for the H460^{C} FSP1^{KO} cells but not the H460^{C} Cas9 cells, validating the ability of our screening approach to detect synthetic lethal relationships.

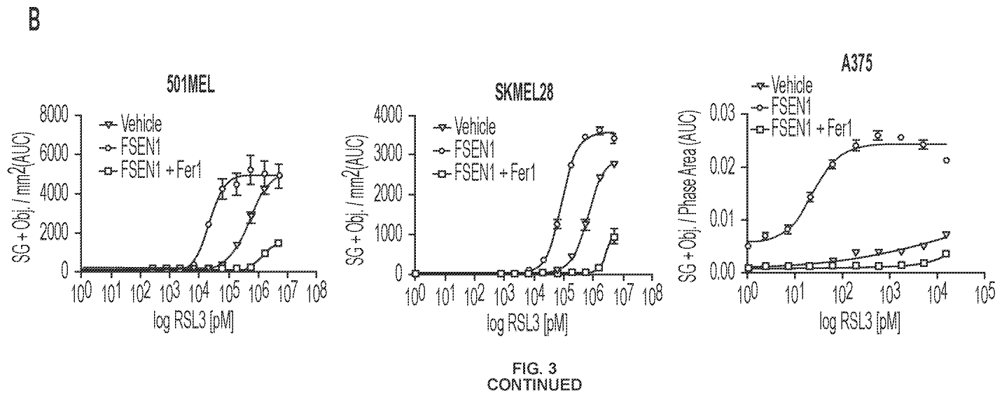

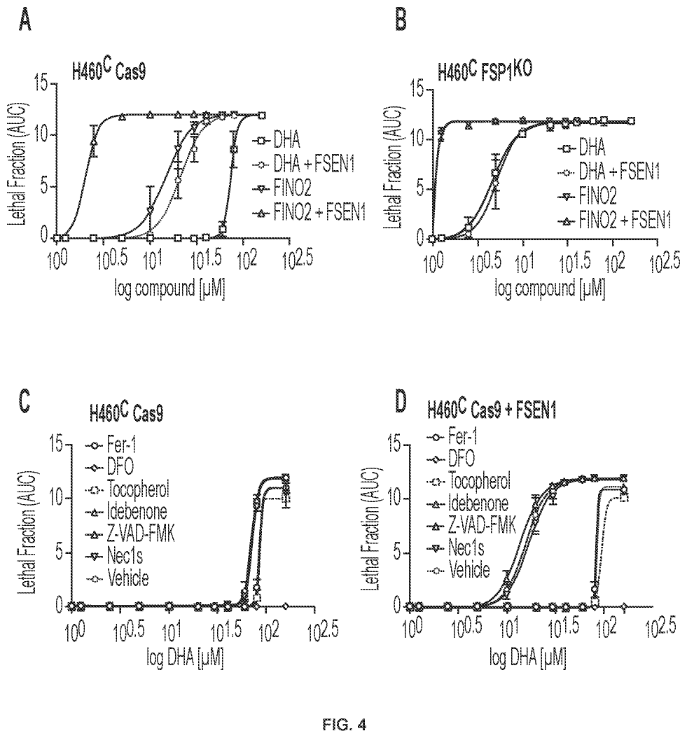

Several compounds selectively induced cell death in the FSP1KO cells, including DHA. DHA is a sesquiterpene lactone compound that has been widely used as an antimalarial and has also been explored as an anti-cancer therapeutic. The endoperoxide bridge within DHA is known to react with ferrous iron and stimulate the formation of toxic free radicals. In addition, DHA was recently shown to induce ferroptosis in multiple cancer types (see, e.g., Contreras, F.-X., et al. Biophys J 88, 348-359 (2005); Zhang, et al. Cancer Letters 381, 165-175 (2016); Nie, J. et al. J Cancer Res Clin Oncol 144, 2329-2337 (2018); and Yi, R., et al. Bioscience Reports 40, BSR20193314 (2020)). Similar to DHA, the ferroptosis inducer FINO2 contains an endoperoxide bridge that oxidizes iron, increases lipid peroxidation, and induces ferroptosis. FSEN1 treatment and FSP1 KO strongly sensitized H460^{C} cells to cell death induced by both DHA and FINO2 (FIG. 4A,B). FSEN1 had no additional sensitizing effect in the FSP1KO cells, indicating that the ability of FSEN1 to sensitize cells to DHA and FINO2 induced cell death is due to its on target inhibition of FSP1. DHA and FSEN1 treatment together induced cell death (DHA EC50=21.3 UM) that was strongly suppressed by ferroptosis inhibiting radical trapping antioxidants (Fer-1, tocopherol, idebenone) and completely suppressed by DFO (FIG. 4C,D).

To characterize the synergy between DHA, RSL3, and FSEN1, a checkerboard dose-response matrix of DHA (0-80 μM) and RSL3 (0-15 μM) in the presence of increasing doses of FSEN1 (0, 0.05, 0.5, 5 μM) was performed, and quantified synergy potency using a computational zero interaction potency (ZIP) modeling system. The ZIP synergy scoring system defines compound synergy as a value >10, additive effects-10<0<10, and antagonistic effects <0. The combinations of FSEN1 and RSL3 (ZIP score=38.28) and FSEN1 and DHA (ZIP score=26.45) exhibited strong synergy. In contrast, DHA and RSL3 showed little synergy (ZIP score=4.11) and a stronger synergy score was observed with all three compounds (ZIP score=52.69). Together, these data indicate that FSP1 inhibition increases cancer cell sensitivity to endoperoxide-containing ferroptosis inducers, including the FDA approved drug DHA. Moreover, these findings demonstrate the potential value of combinatorial therapeutic strategies that combine FSP1 inhibitors with additional ferroptosis inducers to overcome the multitude of cancer ferroptosis defenses.

What is claimed is:

1. A method of inhibiting ferroptosis suppressor protein 1 (FSP1) comprising contacting FSP1 with an FSP1 inhibitor in an amount effective to inhibit FSP1, wherein the FSP1 inhibitor is a compound as shown in Table A.

2. The method of claim 1, wherein the FSP1 inhibitor is

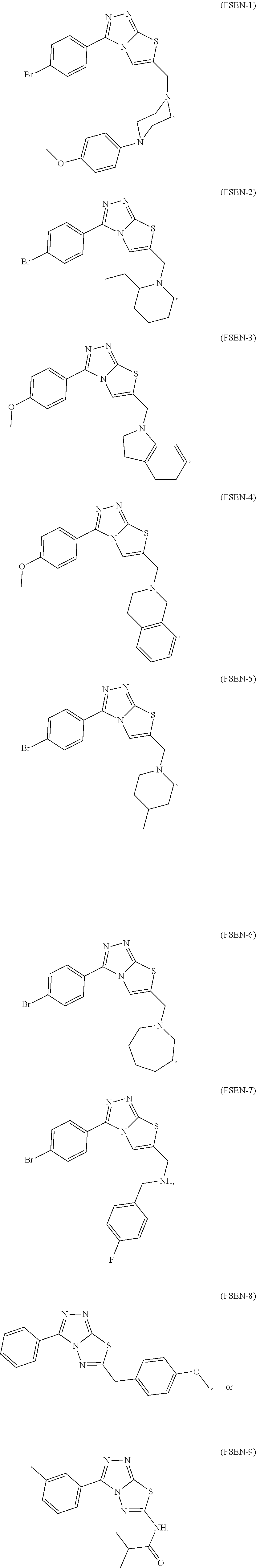

3. A method for treating cancer in a subject suffering therefrom comprising administering to the subject a therapeutically effective amount of an FSP1 inhibitor, wherein the FSP1 inhibitor is a compound as shown in Table A.

4. The method of claim 3, wherein the FSP1 inhibitor is

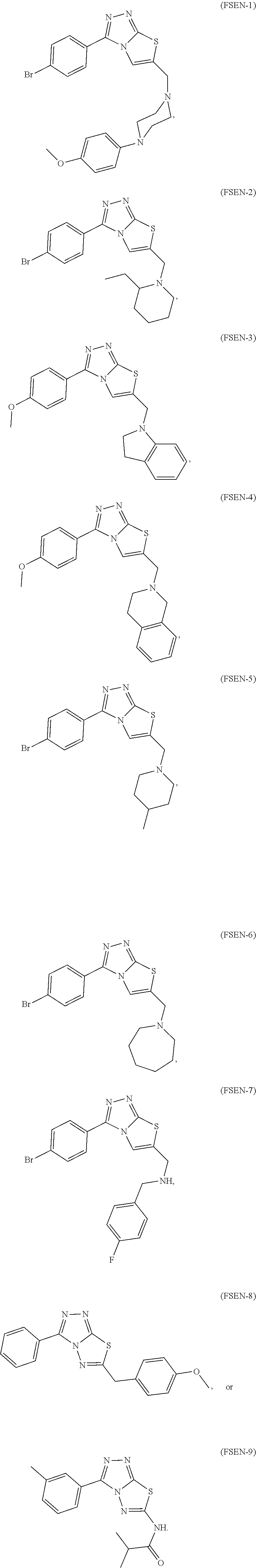

5. The method of claim 3 or 4, further comprising administering to the subject a second therapeutic agent or regimen.

6. The method of claim 5, wherein the second therapeutic agent or regimen comprises radiation, photodynamic therapy, or both.

7. The method of claim 5, wherein the second therapeutic agent is a ferroptosis inducer, including a GPX4 inhibitor, a system xc-inhibitor, an inhibitor of glutathione synthesis, or an endoperoxide.

8. The method of claim 7, wherein the GPX4 inhibitor is DPI3, DPI4, DPI6, DPI7, DPI8, DPI9, DPI10, DPI12, DPI13, DPI15, DPI17, DPI18, DPI19, FIN56, JKE-1674, JKE-1716, ML162, ML210, RSL3, FINO2, Altretamine, NSC144988, or Withaferin A

9. The method of claim 7, wherein the system xc-inhibitor is erastin, erastin2, imidazole ketone erastin, piperazine erastin, DPI2, RSL5, glutamate, sulfasalazine, or sorafenib.

10. The method of claim 7, wherein the inhibitor of glutathione synthesis is buthionine sulfoximine or cyst (e) inase.

11. The method of claim 7, wherein the endoperoxide is artemisinin, dihydroartemisinin (DHA), artemether, arteether, artesunate, artelininic acid, artesunate, artelinate, artemisone, 3-artesanilide, artefenomel, FINO2, or FINO3.

12. The method of any one of claims 3 to 11, wherein the cancer is a ferroptosis-resistant cancer or a GPX4-inhibitor-resistant cancer.

13. The method of any one of claims 3 to 12, wherein the subject had previously undergone a prior therapy.

14. The method of claim 13, wherein the prior therapy was one that triggers oxidative lipid damage.

15. The method of claim 13 or 14, wherein the prior therapy was one or more of photodynamic therapy, radiation, treatment with a GPX4 inhibitor, treatment with an inhibitor of glutathione synthesis, treatment with a system xc-inhibitor, or treatment with an ubiquinone synthesis pathway inhibitor.

16. The method of any one of claims 3 to 15, wherein the cancer is lung cancer, liver cancer, glial cancer, bone cancer, connective tissue cancer, pancreatic cancer, neuroblastoma, colorectal cancer, astrocytoma, adenocarcinoma, breast cancer, brain cancer, liver cancer, kidney cancer, skin cancer, or intestinal cancer.

17. The method of any one of the claims 3 to 16, wherein the cancer expresses FSP1.
